(19) United States
(12) Patent Application Publication
(10) Pub. No.: US 2021/0369153 A1
(43) Pub. Date: Dec. 2, 2021

(54) QUALITY ASSURANCE OF COLLECTED INTERSTITIAL FLUID SAMPLES

(71) Applicant: University of Cincinnati, Cincinnati, OH (US)

(72) Inventors: Jason Charles Heikenfeld, Cincinnati, OH (US); Andrew Jajack, North Canton, OH (US)

(21) Appl. No.: 17/285,240

(22) PCT Filed: Nov. 13, 2019
(86) PCT No.: PCT/US2019/061078

§ 371 Date: Apr. 14, 2021

Related U.S. Application Data
(60) Provisional application No. 62/760,545, filed on Nov. 13, 2018.

(51) Int. Cl. A61B 5/145; A61B 5/1473; A61N 1/30

(52) U.S. Cl. CPC A61B 5/14546; A61B 5/14514; A61B 2562/046; A61N 1/30; A61B 5/1473

(57) ABSTRACT

An interstitial fluid sampling device with mechanical, electrical, and/or chemical components to mitigate sample quality issues and/or measure the quality of collected samples to alert of and/or correct for quality issues. The device includes at least one sensor that is specific to an analyte in the interstitial fluid, at least one wicking component, and at least one component to ensure the quality of collected interstitial fluid.

### CROSS-REFERENCE TO RELATED APPLICATION

This application claims priority to, and the benefit of the filing date of, U.S. Patent Application No. 62/760,545, filed on Nov. 13, 2018, the disclosure of which is incorporated by reference herein in its entirety.

### FIELD OF THE INVENTION

The present invention is generally directed to a device for sampling interstitial fluid, and more specifically to a device that mitigates quality issues with collected interstitial fluid samples.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Interstitial fluid sensing technologies have enormous potential for applications ranging from athletics, to neonatology, to pharmacological monitoring, to personal digital health, to name a few applications. Interstitial fluid contains many of the same biomarkers, chemicals, or solutes that are carried in blood and can provide significant information enabling one to diagnose ailments, health status, toxins, performance, and other physiological attributes even in advance of any physical sign. Interstitial fluid is more directly coupled to blood unlike other biofluids, such as sweat, tears, and urine, where biomarkers must first diffuse through more restrictive tissue layers for reaching those biofluids. Interstitial fluid is advantageous since the concentration of analytes such as metabolites, waste products, signaling molecules, hormones, or pharmaceutical agents may be more clinically-relevant than systemic, blood concentrations. Furthermore, other parameters, attributes, solutes, or features on, near, or beneath the skin can be measured to further reveal physiological information.

Interstitial fluid surrounds cells and functions to exchange nutrients, waste, and signaling molecules between cells and blood. While it is found in tissue systems throughout the body, interstitial fluid in the epidermis and dermis is most commonly used for sensing applications due to its accessibility. Since interstitial fluid is not secreted or excreted from the body, methods of penetrating the skin barrier are necessary for collection. The skin barrier consists of the intracellular lipids of stratum corneum and tight junctions of the upper viable epidermis, the stratum granulosum. Methods of porating the skin, including using lancets, blades, microprojections/microneedles, syringe needles, split needles, blisters, liquid or power jets, lasers, heat, abrasion, ultrasound, or iontophoresis are reviewed by David Cunningham in “Human Body to Device Biofluid Transfer,” Encyclopedia of Microfluidics and Nanofluidics (2015): 1301-1312. The most common of these methods involve hollow needles of either macro or micro scale in single or arrayed configurations. After penetrating the barrier, interstitial fluid can then be removed through via the hollow channel(s).

The common macro/micro-needle(s)-based approach faces several challenges that relate to the quality of collected samples. Contaminants on the surface of the skin, such as dirt, debris, sweat, oils, or microbes, may be drawn into deeper layers of the skin causing unintended negative reactions or into the hollow channels themselves causing interference with downstream assays or sensors. Incomplete penetration into the desired layer of skin may allow the some or all of the hollow channels to come into contact with sweat or diluting fluids such as water from showers, rain, or swimming. When these fluids mix with the interstitial fluid sample, the resulting concentration may be diluted to an unpredictable extent, limiting the clinical relevance of the sample. In addition, these fluids may also contain interfering components such as detergents, acids/bases, or salts that may interfere with downstream assays or sensors. These fluids may also contain target analytes, negatively impacting the clinical relevance of the sample.

Interstitial fluid sensing has not emerged into its fullest opportunity and capability for biosensing, especially for continuous or repeated biosensing or monitoring. However, with proper application of technology, interstitial fluid can be made to actually outperform all other biofluids in providing reliable, clinically-relevant information.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of mechanical elements, chemicals, materials, sensors, electronics, microfluidics, algorithms, computing, software, systems, and other features or designs, in a manner that affordably, effectively, conveniently, intelligently, or reliably collects and senses interstitial fluid. With such a new invention, interstitial fluid sensing could become a compelling new paradigm as a biosensing platform.

The disclosed invention provides an interstitial fluid sampling device with mechanical, electrical, and/or chemical components to mitigate sample quality issues and/or measure the quality of collected samples to alert of and/or correct for quality issues.

One aspect of the present invention provides device for sensing interstitial fluid on skin, including at least one sensor that is specific to an analyte in interstitial fluid, at least one wicking component, and at least one component to ensure the quality of collected interstitial fluid.

Another aspect of the present invention provides a method of preventing outside contamination sources from mixing with a collected interstitial fluid sample. The method includes applying at least one blocking agent to either or both of (a) at least one surface of a device for collecting interstitial fluid, or (b) the skin of a subject from which interstitial fluid is to be collected. The device for collecting interstitial fluid is brought into contact with the skin of the subject, and interstitial fluid is collected from the subject via the device.

Another aspect of the present invention provides a method of delivering an agent to the skin of a subject from which interstitial fluid is to be collected. The method includes applying at least one agent to at least one surface of a device for collecting interstitial fluid, bringing the device for collecting interstitial fluid into contact with the skin of the subject, and delivering the at least one agent to the skin via iontophoresis or via sub-dermal delivery.

Another aspect of the present invention provides a method of assessing the quality of an interstitial fluid sample. The method includes collecting an interstitial fluid sample from a subject, and measuring the fraction of outside contaminants present in the collected interstitial fluid sample.

Another aspect of the present invention provides a method of increasing effectiveness of penetration of skin for sample collection. The method includes stiffening the skin of a subject, and bringing a device for collecting a sample into contact with the skin.

These and other aspects will be described in greater detail below in the detailed description, and with respect to the drawings.

### BRIEF DESCRIPTION OF THE DRAWINGS

The objects and advantages of the disclosed invention will be further appreciated in light of the following detailed descriptions and drawings in which:

FIG. 1A is a top view of an embodiment of an interstitial fluid sensing device.

FIG. 1B is a cross-sectional view of an embodiment of an interstitial fluid sensing device with call-out region identified.

FIG. 2A is a cross-sectional view of an embodiment of said call-out region of FIG. 1B prior to insertion into the skin including a blocking agent.

FIG. 2B is a cross-sectional view of an embodiment of said call-out region of FIG. 1B after insertion into the skin including a blocking agent.

FIG. 3 is a cross-sectional view of an embodiment of said call-out region of FIG. 1B including a delivery component.

FIG. 4 is a cross-sectional view of an embodiment of said call-out region of FIG. 1B including a delivery component.

FIG. 5 is a cross-sectional view of an embodiment of said call-out region of FIG. 1B including electrodes to measure the fraction of outside contaminants in the collected interstitial fluid sample.

FIG. 6 is a cross-sectional view of an embodiment of said call-out region of FIG. 1B including electrodes to measure the fraction of outside contaminants in the collected interstitial fluid sample.

FIG. 7A is a top view of an embodiment of an interstitial fluid sensing device including electrodes to measure the fraction of outside contaminants in the collected interstitial fluid sample, and having a call-out region identified.

FIG. 7B is a top view of said call-out region of FIG. 7A.

FIG. 7C is a cross-sectional view of said call-out region taken along line 7C-7C of FIG. 7B.

FIGS. 8A and 8B are cross-sectional views of an embodiment of an interstitial fluid sensing device demonstrating the viscoelastic properties of skin.

FIG. 9A is a top view of an embodiment of a skin stiffening device.

FIG. 9B is a cross-sectional view of the embodiment of the skin stiffening device of FIG. 9A.

FIG. 10 is a cross-sectional view of an embodiment of a skin stiffening device.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As used herein, an “interstitial fluid sensing component” is any component or material that is capable of sensing interstitial fluid, a solute in interstitial fluid, a property of interstitial fluid, a property of skin due to interstitial fluid, or any other thing to be sensed that is in relation to interstitial fluid. Interstitial sensing components can include, for example, one or multiple sensors such as electrochemical, potentiometric, amperometric, impedance, optical, mechanical, or other mechanisms known by those skilled in the art. An interstitial fluid sensing component may also include supporting materials or features for additional purposes, with non-limiting examples including local-buffering of sensor electronic signals or additional components for interstitial fluid management such as microfluidic materials.

As used herein, the term “analyte-specific sensor” or “sensor specific to an analyte” is a sensor specific to an analyte and performs specific chemical recognition of the analyte's presence or concentration (e.g., ion-selective electrodes, enzymatic sensors, electrically based aptamer sensors, etc.). For example, sensors that sense impedance or conductance of a fluid, such as biofluid, are excluded from the definition of “analyte-specific sensor” because sensing impedance or conductance merges measurements of all ions in biofluid (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

As used herein, “measured” can imply an exact or precise quantitative measurement and can include broader meanings such as, for example, measuring a relative amount of change of something. Measured can also imply a binary measurement, such as ‘yes’ or ‘no’ type measurements.

As used herein, “outside contamination sources” refer to any fluid or solute that may interfere with assay or sensor performance or cause measurements from said assays or sensors to not reflect the target specimen. Outside contamination sources can include bodily fluids such as sweat, sebum, and other skin oils as well as external fluids such as shower water, bath water, rain water, and water from recreational activities such as swimming Outside contamination sources can also refer to contaminants that end up in the fluid or solutes themselves. For example, dirt and debris on the surface of the skin would be included in this definition. Additionally, microbes may produce or consume target analytes, and so are included in the definition.

Some embodiments of the disclosed invention utilize adhesives to hold the device near the skin, but devices could also be held by other mechanisms that hold the device secure against the skin, such as a strap or embedding in a helmet or article of clothing. Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: an interstitial fluid sensor; an impedance sensor; an interstitial fluid volume sensor; and a solute generation rate sensor. Certain embodiments of the disclosed invention show sub-components of what would be interstitial fluid sensing devices with more sub-components needed for use of the device in various applications, which are obvious but not necessarily critical to inventive step (such as a battery, or a counter electrode for iontophoresis), and for purpose of brevity and focus on inventive aspects are not explicitly shown in the diagrams or described in the embodiments of the disclosed invention.

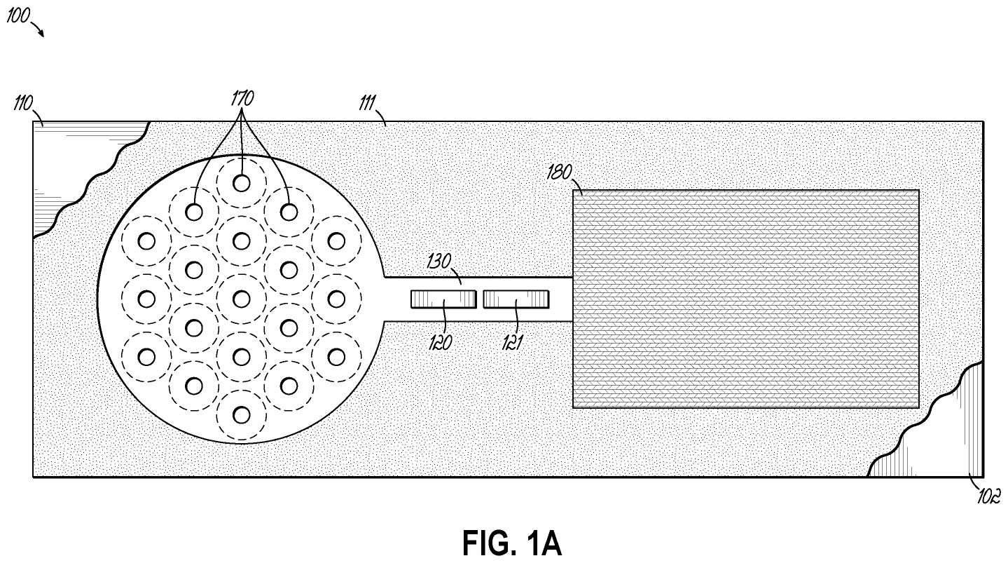

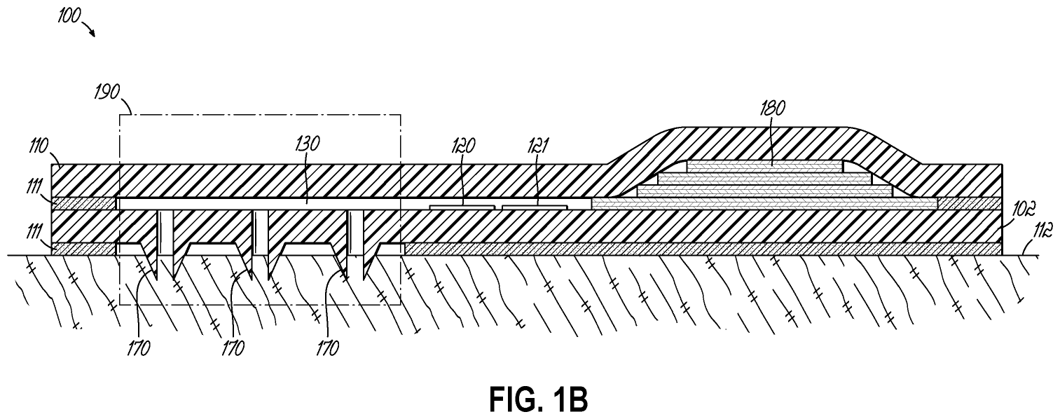

With reference to FIGS. 1A and 1B, an interstitial fluid sensing device 100 includes a lower substrate 102 patterned with sharp protrusions 170, an upper substrate 110, a sample channel 130, one or more sensing components 120, 121, and a wicking component 180. The sample channel 130 may be formed by a space between the lower and upper substrates 102, 110 with the edges of the lower and upper substrates being sealed with an optional adhesive layer 111. Further, the lower substrate 102 may include an optional adhesive 111 suitable for adhering the device 100 to the skin 112. The lower and upper substrates 102, 110 may be flexible or semi-rigid plastic film (e.g., PET) or a textile. Lower substrate 102 carries at least one sensing component 120, 121, which may be, for example, an electrical impedance antibody sensor for cortisol or an electrochemical sensor for glucose.

In an example of device operation, the device 100 is first placed on the skin 112, then the sharp protrusions 170 of the lower substrate 102 penetrate the barrier layers of the skin 112, allowing access to interstitial fluid. The sample channel 130 in combination with the wicking component 180 is capable of generating the pressure gradient necessary to pump interstitial fluid across the sensing components 120, 121. In addition to or alternatively, channel 130 can be filled with a fluid such as interstitial fluid and analytes able to diffuse from interstitial fluid in the dermis to the sensors 120, 121.

With further reference to FIG. 1B, sensing components may sense similar or different components of the sample fluid or properties thereof and do not necessarily need to be of the same type. In an example, the wicking component 180 may be paper or a water absorbing polymer such as, for example, a hydrogel. The sharp protrusions 170 may exist along the entire length and width of the surface of the lower substrate 102 that contacts the skin 112, or may be isolated into a specific region as shown as an example in FIG. 1A. The shape, diameter, abundance, angle, symmetry, spacing, length, and other properties of the design of the sharp protrusions 170 may be selected based on the region of the body where the device will used (e.g., regions of the body where the barrier layers of the skin are thicker might require longer protrusions than regions of the body were the barrier layers of the skin are thinner). The capacity of the wicking component may be selected to allow the device to be used for a specified duration. As an example shown in FIG. 1A, the upper substrate 110 and associated optional adhesive 111 may be oversized, i.e., extend beyond the perimeter of the lower substrate 102, to provide greater protection from outside contamination sources. Further aspects of the invention focus on region 190 of device 100 as shown in FIG. 1B.

With further reference to FIG. 1B, another example interstitial fluid sensing device 100 includes sensing components 120, 121 capable of chemically measure the fraction of outside contaminants in the collected interstitial fluid sample. Measuring the fraction of sweat in the collected interstitial fluid sample is an example of an outside contaminant that can be measured by sensing components 120, 121, or by another sensor in another location of device 100 (not shown). In this example, at least one sensor, measures a component in sweat having a stable or predictable concentration range such as lactate, potassium ions or albumin; measures a component in sweat with narrow concentration range such as dermcidin; or measures a component in sweat with larger concentration range such as salinity. Obviously, using a more stable component is advantageous to provide more accurate fractions.

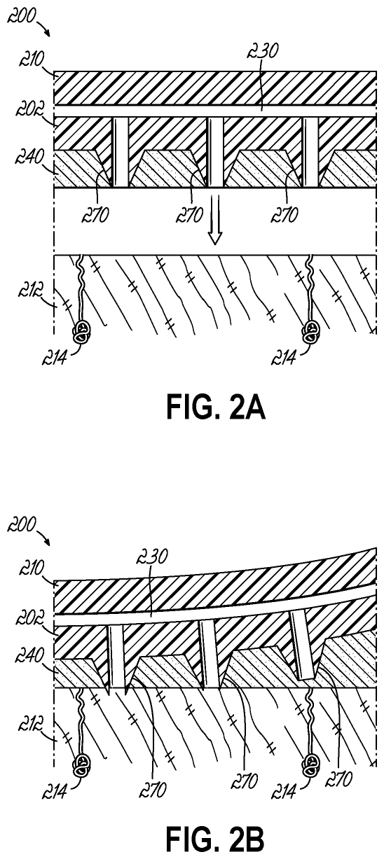

With reference to FIGS. 2A and 2B, an interstitial fluid sensing device 200 includes a lower substrate 202 patterned with sharp protrusions 270, an upper substrate 210, and a sample channel 230. The device 200 of this embodiment includes a blocking agent 240 that may be coated onto lower substrate 202 (which corresponds to lower substrate 102 of FIGS. 1A and 1B), or may be directly applied to the skin 212. Potential blocking agents 240 include hydrophobic materials, such as petroleum jelly or cosmetic-grade silicon oil, or adhesives/resins that may be activated or cured when exposed to water or certain pH values, salt concentrations, or temperatures. Activating or curing agents may be precoated on the skin 212, or embedded in dry form into the adhesive/resin coating of the lower substrate 202 and, when exposed to water, become aqueous and react with adhesive/resin coating. In embodiments, the blocking agent 240 extends over the and seals the openings in the sharp protrusions 270 of the lower substrate 202 to provide a hermetic seal for the device 200 when the sharp protrusions 270 are not penetrating the skin, but allows interstitial fluid to enter the sharp protrusions 270 when the protrusions 270 have penetrated the skin 212. In another embodiment, the blocking agent 240 does not extend over the openings in the sharp protrusions 270, but provides a hermetic seal in between the protrusions 270. The hermetic sealing provided by these blocking agents 240 prevents outside contamination sources such as sweat 214 from mixing with the collected interstitial fluid sample when some protrusions 270 are not penetrating the skin 212.

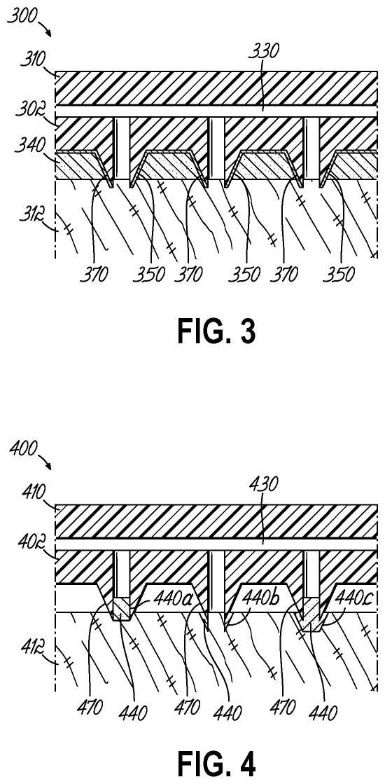

With reference to FIG. 3, an interstitial fluid sensing device 300 includes a lower substrate 302 patterned with sharp protrusions 370, an upper substrate 310, and a sample channel 330. The device 300 of this embodiment includes a delivery component 340 that may be coated onto the lower substrate 302 or directly applied to the skin 312. The delivery component 340 may include a hydrogel or other absorbent material capable of containing agents to reduce contamination or improve user experience. Agents to reduce contamination include agents that prevent sweating such as anti-cholinergics (e.g., glycopyrrolate, poldine methylsulfate, etc.) or agents that sterilize the skin such as alcohols (e.g., ethanol, isopropyl alcohol, etc.) or antimicrobial agents. Agents to improve user experience include anti-inflammatory agent (e.g., diphenhydramine, hydrocortisone), anti-itch agents (e.g., doxepin), or anti-pain agents (e.g., lidocaine). Agents within the delivery component 340 may be delivered actively or passively. Active delivery of charged agents is possible using iontophoresis. Many of the agents listed above including glycopyrrolate and poldine methylsulfate are charged. Iontophoresis is the movement of a charged species in response to an applied electric current. Substrate 302 may be made of a electrically conducting material, such as a conducting carbon material or a metal material, and used as an iontophoresis electrode or a separate conductive component 350, such as an electrically conducting material, may applied to the surface of the lower substrate 302 adjacent the skin 312 instead. For the embodiments utilizing a iontophoresis, the electrically conducting material is electrically coupled to a power source, such as a battery, and optionally, to circuitry to control the operation of the iontophoretic components. Passive delivery relies on the diffusivity of the agent. Some agents can penetrate the skin barrier and may be applied topically. Other agents may need assistance to move through the skin barrier.

As an example, in FIG. 4, an interstitial fluid sensing device 400 includes a delivery component 440 that is found inside the protrusions 470 as at 440a, coating the protrusions 470 as at 440b, or both as at 440c. As the protrusions 470 break through the skin barrier 412, so too will the agents contained within the delivery component 440.

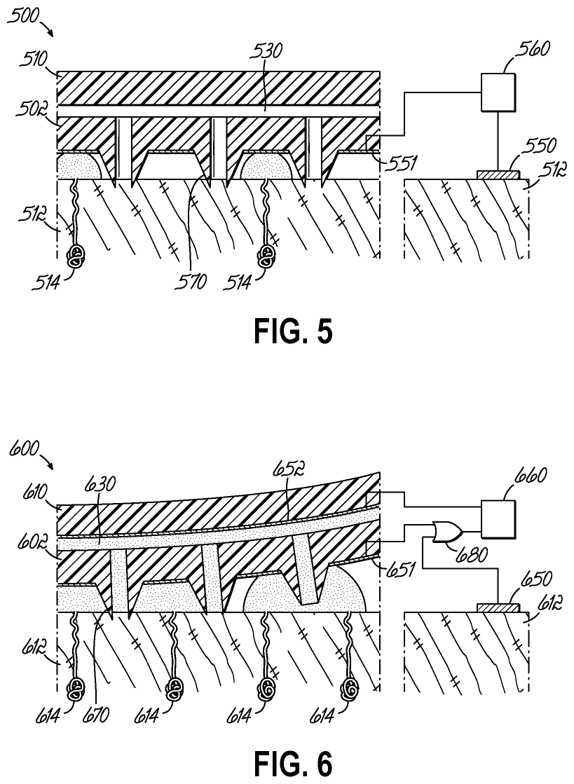

With reference to FIG. 5, an interstitial fluid sensing device 500 includes a lower substrate 502 patterned with sharp protrusions 570, an upper substrate 510, and a sample channel 530. The device 500 of this embodiment includes an electrode 551 that may or may not cover the entire outer surface of the lower substrate 502 connected to an impedance measuring component 560. In embodiments in which substrate 502 is made of conductive material, the electrode 551 may not be included. A counter electrode 550 completes the circuit and is simply shown here as being separately affixed to the skin 512. However, the counter electrode 550 can be directly incorporated into the device and may include adhesive and sealing substrates. In cases where an outside contamination source such as sweat from sweat gland 514 in the skin 512 makes contact with the electrode 551, the impedance of the circuit may change. In some cases, the resistive component of impedance will dominate and so measurement component 560 may be simplified to just measure resistance. In other cases, the capacitive component of impedance will dominate and so measurement component 560 may be simplified to just measure capacitance.

With reference to FIG. 6, an interstitial fluid sensing device 600 includes a lower substrate 602 patterned with sharp protrusions 670, an upper substrate 610, and a sample channel 630. The device 600 of this embodiment includes an electrode 652 that may or may not cover the entire lower surface of the upper substrate 610 that forms the channel 630. The upper substrate electrode 652 connected to an impedance measuring component 660. In embodiments in which the upper substrate 610 is made of conductive material, the electrode 652 may not be necessary. A counter electrode 650 completes the circuit and is shown here as being separately affixed to the skin 612. However, the counter electrode 650 may also be an electrode 651 that may or may not cover the lower surface of the lower substrate 602. The device 600 of this embodiment may also include a logic gate (an OR gate in FIG. 6) 680 positioned between electrodes 650, 651 and impedance measuring component 660. In cases where an outside contamination source such as sweat from sweat gland 614 enters channel 630 and makes contact with the electrode 652, the impedance of the circuit may change.

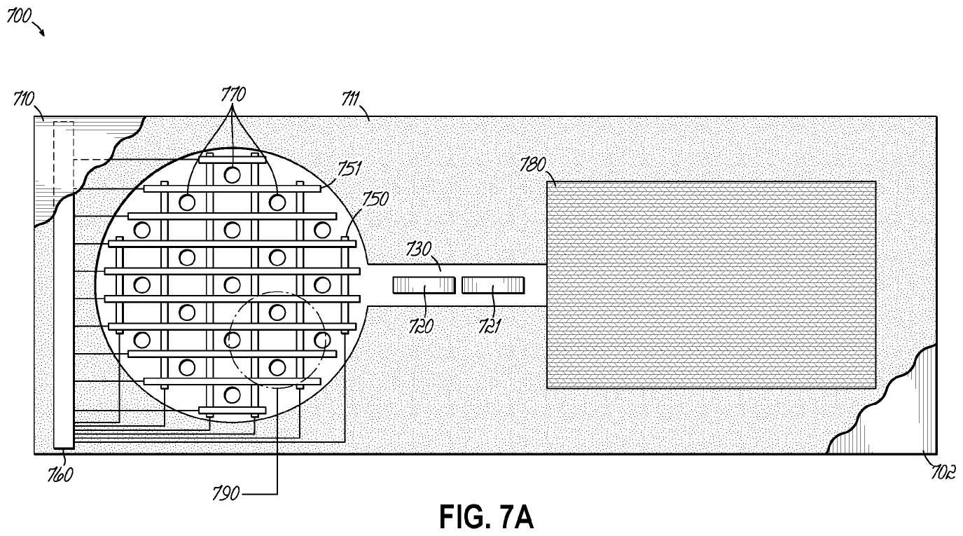

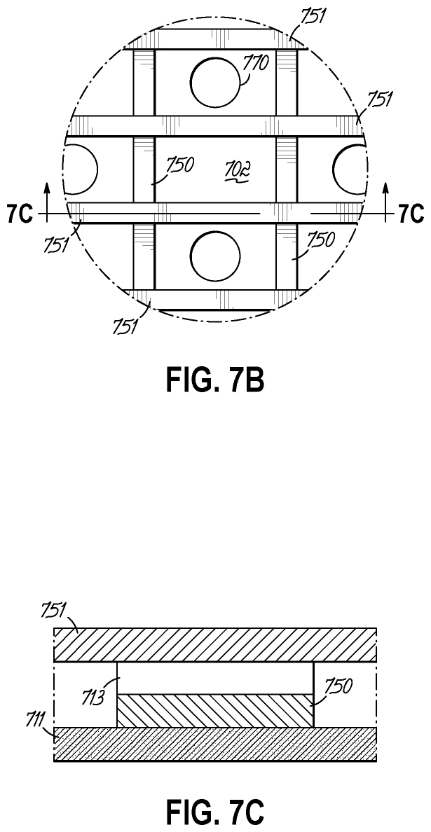

With reference to FIGS. 7A, 7B, and 7C, an interstitial fluid sensing device 700 includes a lower substrate 702 patterned with sharp protrusions 770, an upper substrate 710, a sample channel 730, one or more sensing components 720, 721, and a wicking component 780. The device 700 of this embodiment includes at least one electrode 750 that covers at least a portion, and in some embodiments, covers the entire surface of the upper substrate 710 in a repeated array connected to an impedance measuring component 760. If an adhesive layer 711 is present, the electrode 750 may be affixed to the adhesive layer 711. The counter electrode 751 may be as described previously, or a repeated array similar to that of electrode 750. Electrode 750 and counter electrode 751 as seen in call-out 790, may be arranged in crisscross pattern. To prevent shorting, a spacer layer 713 between electrode 750 and counter electrode 751 is present and may be made from an electrically insulating material such as a plastic (PET) or adhesive (silicone-based) as seen in FIG. 7C. The lower substrate 702 and protrusions 770 are shown for perspective in the call-out region 790 (see FIG. 7B). Multiple electrodes 750 and 751 can then be utilized to measure the presence of interstitial fluid and or the presence of sweat by techniques such as electrical impedance or other suitable techniques. For example, if any of the protrusions 770 are not fully inserted into the skin, then they may not initially receive interstitial fluid, or later they more easily receive sweat, either being detectable locally for the effected protrustions by scanning through the electrodes 750, 751 to measure electrical impedance. Therefore the present invention also includes at least one component to measure the presence of contamination to a single or multiple protrusions and/or incomplete insertion of a single or multiple protrusions into the skin.

Besides the chemical and electrical methods described above, mechanical and optical methods of measure the fraction of outside contaminants in the collected interstitial fluid sample are possible but not described herein.

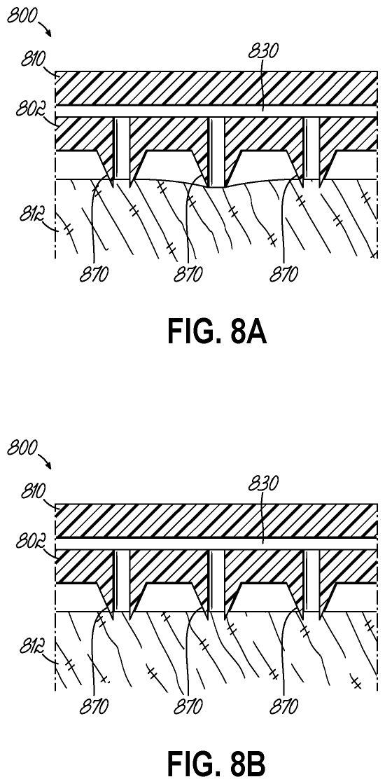

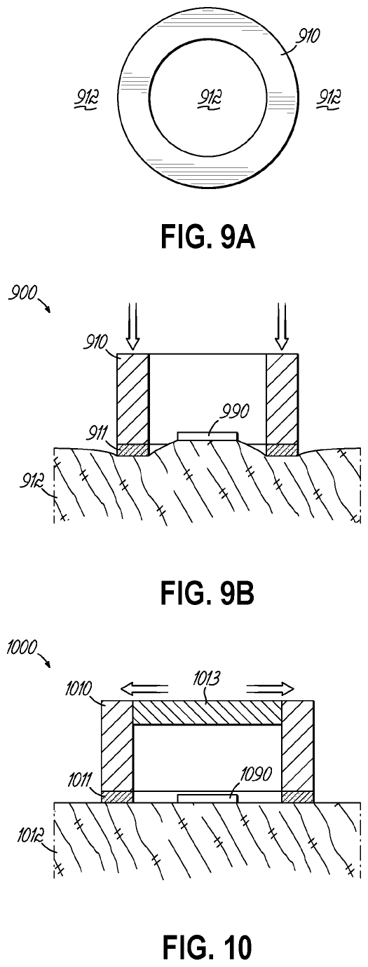

With reference to FIGS. 8A and 8B, an interstitial fluid sensing device 800 includes a lower substrate 802 patterned with sharp protrusions 870, an upper substrate 810, and a sample channel 830. The device 600 of this embodiment. As seen in FIG. 8A, the skin 812 is a viscoelastic material that may conform around protrusions 870. Stiffening the skin prior to insertion may increase penetration effectiveness as shown in FIG. 8B. With reference to FIGS. 9A and 9B, a skin stiffening device or component 900 includes a ring-shaped substrate 910 that when depressed stretches the skin 912 prior to insertion. An optional adhesive layer 911 may be included on the lower surface of the skin stiffening device 900 to prevent the device 900 from sliding. In addition, an interstitial fluid sensing device 990 may be integrated directly or separately as shown in FIG. 9B. With reference to FIG. 10, a skin stiffening device 1000 includes a ring-shaped substrate 1010 that when depressed stretches the skin 1012 prior to insertion as a result of the elongation of an expandable member 1013. An optional adhesive layer 1011 may be included to prevent the device 1010 from sliding. In addition, an interstitial fluid sensing device 1090 may be integrated directly or separately as shown in FIG. 10.

Prior to insertion, the skin may contain dirt, debris, initial skin oils, initial sweat components, and some microbes. Preparing the skin using physical and chemical sterilization methods may improve sample quality. Physical removal methods include wiping, tape striping, air/vacuum, etc. Chemical sterilization methods include applying alcohols (ethanol, isopropyl alcohol, etc.) or antimicrobials directly to the surface of the skin.

While the present invention has been disclosed by reference to the details of preferred embodiments of the invention, it is to be understood that the disclosure is intended as an illustrative rather than in a limiting sense, as it is contemplated that modifications will readily occur to those skilled in the art, within the spirit of the invention and the scope of the amended claims.

What is claimed is:

1. A device for sensing interstitial fluid on skin, comprising:
at least one sensor that is specific to an analyte in interstitial fluid; and at least one component to ensure the quality of collected interstitial fluid.

2. The device of claim 1, wherein the at least one component to ensure quality includes at least one blocking agent.

3. The device of claim 2, wherein the at least one blocking agent includes a hydrophobic material.

4. The device of claim 2, wherein the at least one blocking agent is selected from adhesives or resins that are activated upon contact with an outside contamination source.

5. The device of claim 1, wherein the at least one component to ensure quality includes at least one delivery component.

6. The device of claim 5, wherein the at least one delivery component includes at least one agent selected from an agent to reduce contamination; an agent to reduce pain; an agent to reduce skin irritation; an agent that prevents sweating; an agent that sterilizes the skin; an anti-inflammatory agent; an anti-itch agent; an anti-pain agent; and combinations thereof.

7. The device of claim 1, wherein the at least one component to ensure quality includes at least one sensor specific to a component of an outside contaminant.

8. The device of claim 1, wherein the at least one component to ensure quality includes at least one electrode connected to at least one impedance measuring component.

9. The device of claim 8, wherein the at least one electrode is on an external surface of the device and may come into contact to an outside contamination source.

10. The device of claim 8, wherein the at least one electrode is on an internal surface of the device and may come into contact to an outside contamination source.

11. The device of claim 8, wherein the at least one electrode is in a repeated array and is connected to an impedance measuring component.

12. The device of claim 1, wherein the at least one component to ensure quality includes a substrate that stretches the skin.

13. The device of claim 1, further comprising at least one wicking component.

14. A method of preventing outside contamination sources from mixing with a collected interstitial fluid sample, comprising:
applying at least one blocking agent to: (a) at least one surface of a device for collecting interstitial fluid, or (b) the skin of a subject from which interstitial fluid is to be collected;
bringing the device for collecting interstitial fluid into contact with the skin of the subject; and
collecting interstitial fluid from the subject via the device.

15. The method of claim 14, wherein the device includes at least one protrusion configured to penetrate the skin when brought into contact therewith, the at least one protrusion having an opening for passage of interstitial fluid, and wherein applying the at least one blocking agent to at least one surface of the device provides a seal for the opening when the at least one protrusion is not penetrating the skin.

16. A method of delivering an agent to the skin of a subject from which interstitial fluid is to be collected, comprising:
applying at least one agent to at least one surface of a device for collecting interstitial fluid;
bringing the device for collecting interstitial fluid into contact with the skin of the subject; and
delivering the at least one agent to the skin via iontophoresis or via sub-dermal delivery.

17. The method of claim 16, wherein the device includes at least one protrusion configured to penetrate the skin when brought into contact therewith, and when delivery of the at least one agent is accomplished via sub-dermal delivery, the at least one agent is applied to said at least one protrusion prior to bringing the device into contact with the skin of the subject.

18. A method of assessing the quality of an interstitial fluid sample comprising:
collecting an interstitial fluid sample from a subject; and
measuring the fraction of outside contaminants present in the collected interstitial fluid sample.

19. The method of claim 18, wherein measuring the fraction of outside contaminants in the collected interstitial fluid sample is performed chemically, electrically, mechanically, optically, or a combination thereof.

20. A method of increasing effectiveness of penetration of skin for sample collection, comprising:
stiffening the skin of a subject; and
bringing a device for collecting a sample into contact with the skin.

21. The method of claim 20, wherein stiffening the skin of the subject is accomplished by stretching the skin.
